SEQUENCE LISTING (9 sequences)

SEQ ID NO: 1 - protein, 1130 aa, Homo sapiens
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 2 - DNA, 20 bp, Artificial Sequence
  Synthetic
tagtccttcccaccccatct

SEQ ID NO: 3 - DNA, 20 bp, Artificial Sequence
  Synthetic
agggaggctctgtctttggt

SEQ ID NO: 4 - DNA, 20 bp, Artificial Sequence
  Synthetic
ttggaggaaaacccagtcag

SEQ ID NO: 5 - DNA, 20 bp, Artificial Sequence
  Synthetic
gctctggaggaggaagtgtg

SEQ ID NO: 6 - DNA, 20 bp, Artificial Sequence
  Synthetic
tggttttgggacccagagta

SEQ ID NO: 7 - DNA, 20 bp, Artificial Sequence
  Synthetic
ggttggggtcattttcactg

SEQ ID NO: 8 - DNA, 20 bp, Artificial Sequence
  Synthetic
ccaacaaaaacccattcagc

SEQ ID NO: 9 - DNA, 20 bp, Artificial Sequence
  Synthetic
gttggggtcattttcactgg

(19) United States
(12) Patent Application Publication
(10) Pub. No.: US 2011/0263609 A1
(43) Pub. Date: Oct. 27, 2011

(54) Methods of Identifying and Treating Individuals Exhibiting NUP214-ABL1 Positive T-Cell Malignancies with Protein Tyrosine Kinase Inhibitors and Combinations Thereof

(76) Inventors: Alfonso Quintas-Cardama Lee, Houston, TX (US); Guillermo Garcia-Manero, Bellaire, TX (US)

(21) Appl. No.: 12/742,360

(22) PCT Filed: Nov. 14, 2008
(86) PCT No.: PCT/US08/83653

§ 371 Date: Feb. 25, 2011

Related U.S. Application Data
(60) Provisional application No. 60/988,290, filed on Nov. 15, 2007.

(51) Int. Cl. A61K 31/497; C07D 417/14; A61P 43/00; C12Q 1/68

(52) U.S. Cl. 514/252.19; 435/6.11; 544/295

(57) ABSTRACT

The invention described herein relates to diagnostic and treatment methods and compositions useful in the management of disorders, for example cancers, involving NUP214-ABL1 positive T-cell malignancies and methods for treating an individual suffering from a NUP214-ABL1 positive T cell malignancy.

This application claims priority to U.S. Provisional Ser. No. 60/988,290 filed Nov. 15, 2007.

### FIELD OF THE INVENTION

The invention described herein relates to diagnostic and treatment methods and compositions useful in the management of disorders, for example cancers, involving NUP214-ABL1 positive T-cell malignancies.

### BACKGROUND OF THE INVENTION

Cancer is the second leading cause of human death next to coronary disease. Worldwide, millions of people die from cancer every year. In the United States alone, cancer causes the death of well over a half-million people annually, with some 1.4 million new cases diagnosed per year. While deaths from heart disease have been declining significantly, those resulting from cancer generally are on the rise and are predicted to become the leading cause of death in the developed world.

BCR-ABL1, a fusion oncogene generated by a reciprocal translocation between Chromosomes 9 and 12, encodes the BCR-ABL1 fusion protein, a constitutively active cytoplasmic tyrosine kinase present in >90% of all patients with chronic myelogenous leukemia (CML), and in 15-30% of adult patients with acute lymphoblastic leukemia (ALL).¹⁻³ Numerous studies have demonstrated that the underlying pathophysiology of CML is the kinase activity of BCR-ABL.^{1,4} The clinical success of the BCR-ABL kinase inhibitor imatinib (Gleevec®) has validated its use in the management of CML.

By contrast, BCR-ABL1 has only been detected anecdotally in T-cell acute lymphoblastic leukemia (T-ALL).^{5,6} Amplification of the ABL1 gene in the absence of the BCR-ABL1 transcript was first reported in 8 patients with T-ALL, appearing as multiple signals by fluorescence in situ hybridization (FISH).⁷ Graux et al identified the formation of episomal structures resulting from the fusion between ABL1 and NUP214 as a novel mechanism of tyrosine kinase activation in cancer.⁸ The NUP214-ABL1 cryptic transcript was detected in 5 (5.8%) of 85 patients with T-ALL and in 3 of 22 T-ALL cell lines screened.⁸ A more recent report has identified the presence of NUP214-ABL1 in 11 (3.9%) of 279 adult patients with T-ALL.⁹

Despite a steady improvement in outcome, the current 5-year event-free survival rate for adult patients with ALL with modern chemotherapy regimens is only 40%.¹⁰ This is unlikely to be significantly improved by intensifying existing chemotherapy regimens, but rather through the targeting of key elements in the pathogenesis of this disease.¹⁰ Therapy with the relatively selective ABL1 tyrosine kinase inhibitor (TKI) imatinib, is associated with remarkable clinical activity in patients with CML.^{8,11}

The discovery of NUP214-ABL1 in T-ALL was made in 2004.⁸ TKIs with enhanced activity against ABL1 kinase and with the ability to override resistance mediated by most identified ABL1 kinase domain mutants have been developed. One such agent, nilotinib (formerly AMN107), is a phenylaminopyrimidine based on the crystal structure of the ABL1 kinase domain in complex with imatinib.¹² Like imatinib, nilotinib binds ABL1 in its inactive conformation, but exhibits 30-fold higher inhibitory activity.¹² Dasatinib is a thiazolylamino-pyrimidine structurally unrelated to imatinib and nilotinib, with potent inhibitory activity against a variety of tyrosine kinases, including ABL1 and Src family kinases (SFKs).¹³⁻¹⁵ Dasatinib poses less stringent conformational requirements, thus binding ABL1 both in its active and inactive conformations, resulting in 1- and 2-log higher potency than nilotinib and imatinib, respectively.¹⁴ The increased activity of nilotinib and dasatinib against ABL1 kinase has translated into remarkable clinical activity in patients with CML in lymphoid blast phase or BCR-ABL1-positive B-ALL.^{16,17}

In view of the discovery of NUP214-ABL1 in T-ALL, there is a need for improved detection and treatment of patients with NUP214-ABL1-positive T-ALL. The invention provided herein satisfies this and other needs.

### SUMMARY OF THE INVENTION

In one embodiment, the present invention provides a method for predicting responsiveness of an individual with a T cell malignancy to treatment with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, which method comprises: screening a biological sample from said individual for the presence of NUP214-ABL1, wherein the presence of NUP214-ABL1 in the biological sample indicates that said individual with the T cell malignancy is predicted to be responsive to treatment with N-(2-chloro-6-methylphenyl)-2-[[6-[4-(2-hydroxyethyl)-1-piperazinyl]-2-methyl-4-pyrimidinyl]amino]-5-thiazolecarboxamide.

In another embodiment, the present invention provides a method of treating an individual suffering from a T cell malignancy, which method comprises: determining whether the individual harbors NUP214-ABL1; and administering a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, to the individual who harbors NUP214-ABL1.

In another embodiment, the present invention provides a kit for use in determining a treatment strategy for an individual with a T cell malignancy, comprising: a means for determining whether the individual harbors NUP214-ABL1; and instructions for use and interpretation of the kit results.

In yet another embodiment, the present invention provides the use of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate or solvate thereof for preparing a medicament for the treatment of a patient with a NUP214-ABL1 positive T cell malignancy.

### BRIEF DESCRIPTION OF THE FIGURES

FIG. 1. Detection of the NUP214-ABL1 rearrangement and survival of patients with ALL expressing NUP214-ABL1. (A) Detection of NUP214-ABL1 transcripts in 3 patients with T cell malignancies and in the T-ALL cell line PEER. The sequences of the encountered NUP214-ABL1 transcripts are shown. (B, C) Marrow specimens from 29 patients were screened for the presence of NUP214-ABL1 by FISH. Images of bone marrow smears are shown using the NUP214/ABL1 red/green fusion probe, consisting of BAC clones RP11-544A12 (green) and RP11-83J21 (red). Cell with fusion amplification (B, arrow) displays multiple intense fusion yellow signals (cohybridization), whereas normal cells each have only two red/greens fusion signals. Samples were analyzed using a Zeiss Axiphot fluorescent microscope including single- and triple-band pass filters. (D) All 29 patients included in the study were uniformly treated with hyperCVAD chemotherapy. Kaplan-Meier survival curves demonstrate no significant differences regarding overall survival between NUP214-ABL1-positive and -negative patients (p=0.34). Experiments were performed as described in Example 1.

FIG. 2. Detection of NUP214-ABL1 minimal residual disease. (A) Patient 2 was diagnosed with NUP214-ABL1-positive T cell lymphoblastic lymphoma expressing a NUP31-a2 rearrangement (lane 2). HyperCVAD therapy resulted in CR by standard morphologic and flow cytometric criteria, which is still ongoing 9 months into maintenance chemotherapy. However, the NUP214-ABL1 transcript can be detected in peripheral blood by nested PCR, thus confirming the presence of residual disease (lane 1). NUP34-a2 transcript in PEER (lane 3) and BE-13 (lane 4) cells is also shown. (B) NUP214-ABL1-positive cells were also demonstrated by FISH using a specific NUP214-ABL1 red/green fusion probe in a bone marrow specimen obtained from Patient 2 at the same time point. Experiments were performed as described in Example 1.

FIG. 3. Viability of PEER and BE-13 cells upon exposure to imatinib, nilotinib, or dasatinib. The viability of the NUP214-ABL1-positive cell lines PEER (A-C) and BE-13 (D-F) was significantly reduced after 72 hours of exposure to increasing concentrations of imatinib, nilotinib, or dasatinib. The concentration of nilotinib and dasatinib required to inhibit the growth of PEER and BE-13 cells by 50 percent (IC₅₀) was significantly lower than the IC₅₀ values for imatinib (F test, p=0.001). By contrast, the NUP214-ABL-negative T-ALL cell line Jurkat (red curve in panel A), was remarkably resistant to imatinib, indicating that the cytotoxicity mediated by these TKIs is not related to a general toxic effect on T cells. Experiments were performed as described in Example 2.

FIG. 4. Induction of apoptosis of NUP214-ABL1-positive PEER cells by imatinib, nilotinib, and dasatinib. (A) Flow cytometry analysis of the proapoptotic effects of imatinib, nilotinib, and dasatinib on PEER cells treated at the respective IC₅₀ concentrations of each compound. A total of 10000 events were analyzed. (B) Percentage of apoptotic PEER cells after treatment with each TKI. Dasatinib therapy was associated with the highest number of apoptotic cells after 48 hours of treatment. (C) Cell cycle analyses on PEER cells treated with dasatinib were performed at 24, 48, and 72 hours by determination of the DNA content. Cells were stained with PI and cell nuclei were analyzed by flow cytometry. Experiments were performed as described in Example 2.

FIG. 5. Nilotinib-induced and dasatinib-induced PARP and caspase cleavage. PEER cells were exposed to 50 nM or 100 nM of nilotinib or dasatinib for 16, 24, and 48 hours. Whole cell lysates were separated on sodium dodecyl sulfate-polyacrylamide gel electrophoresis (SDS-PAGE) and immunoblotting was performed using monoclonal antibodies against PARP, caspase-3 and -9, and BCL-2. Both compounds induced time-dependent cleavage of PARP (85 kDa fragment) as well as proteolytic activation of caspase-3 and caspase-9. Treatment with both TKIs induced decreasing levels of the proapoptotic protein BCL-2 after 24 hours. This effect was more pronounced in PEER cells treated with dasatinib. Treatment with dasatinib 100 nM for 48 hours resulted in undetectable levels of BCL-2. Experiments were performed as described in Example 2.

FIG. 6. Imatinib, nilotinib, and dasatinib inhibit the phosphorylation of signaling elements downstream of NUP214-ABL kinase. PEER cells were treated for 3 hours with imatinib or nilotinib at their respective IC₈₀, IC₅₀, and IC₂₀ concentrations (A) or with nilotinib or dasatinib at 1, 10, 50, and 100 nM (B). Whole PEER cell lysates were prepared, transferred to membranes, and total protein was analyzed by Western blot using anti-CrKL, anti-p-CrKL, anti-STAT5, or anti-p-STAT5. (A) Nilotinib inhibits CrKL phosphorylation more efficaciously than imatinib. (B) Dasatinib inhibits ABL and CrKL phosphorylation more efficaciously than nilotinib. Experiments were performed as described in Example 2.

FIG. 7. In vivo activity of dasatinib against NUP214-ABL1-positive cells. (A) Dasatinib (▪) or placebo () at 15 mg/kg were administered daily to NOD/SCID mice bearing subcutaneous SIL-ALL tumors. Tumor volume was measured on the indicated days, with the mean tumor volume±SEM indicated for each group, each of which consisting of 8 mice. Mice receiving dasatinib exhibited decreased tumor growth compared with those treated with placebo (p=0.02). (B) Kaplan-Meier survival analysis of NOD/SCID mice harboring SIL-ALL xenografts treated with placebo, dasatinib (Das) at 15 mg twice daily, or dasatinib at 30 mg daily. Dasatinib therapy prolonged significantly the survival of mice treated with dasatinib as compared with those treated with placebo (p<0.005). (C) Treatment of marrow lymphoblasts obtained from Patient 1 with dasatinib and nilotinib resulted in remarkable decrease of the phosphorylation of CrKL and STAT5, indicating an inhibitory effect of both TKIs on NUP214-ABL1-positive leukemic T cells. Experiments were performed as described in Example 2.

### DETAILED DESCRIPTION OF THE INVENTION

The present invention provides, inter alia, methods for predicting the responsiveness of an individual with a NUP214-ABL1 positive T-cell malignancy to treatment with dasatinib, nilotinib or a combination of one or more of dasatinib, nilotinib, and another therapy for treating a NUP214-ABL1 positive T cell malignancy. The present invention also provides methods for treating an individual suffering from a NUP214-ABL1 positive T cell malignancy by administering a therapeutically effective amount of dasatinib, nilotinib or a combination of one or more of dasatinib, nilotinib, and another therapy for treating a NUP214-ABL1 positive T cell malignancy. The terms “treating,” “treatment” and “therapy” as used herein refer to curative therapy, prophylactic therapy, preventative therapy, and mitigating disease therapy.

NUP214-ABL1 is a recently identified gene fusion resulting from episomal fusion of the ABL1 gene to the neighboring NUP214 gene.⁹ Graux et al identified 5 different NUP214-ABL1 transcripts among 85 patients with T-ALL who displayed episomal ABL1 overamplification and demonstrated the selective absence of the 5′ end of ABL1 in the amplicon, in concordance with the involvement of ABL1 in the generation of the fusion gene.⁸ Other NUP214-ABL1 genomic presentations have also been demonstrated, including intrachromosomal amplification and 9q34 insertions, which can coexist in the same patient.¹⁸ Recently, the NUP214-ABL1 fusion has been reported in 11 (3.9%) of 279 patients with T-ALL by means of a multiplex RT-PCR approach that included 10 different NUP214 forward primers and 2 ABL1 primers (a2 and a3) to allow amplification of all possible NUP214-ABL1 in-frame transcripts.⁹ NUP214-ABL1 has been identified in 4 human T-ALL cell lines among 22 screened: PEER, SIL-ALL, TALL-104, and in BE-13, a tetraploid subline of PEER.⁸

Despite the multiple possible permutations between ABL1 exons a2 and a3 (encoding the SH3 regulatory domain of ABL1 kinase) and the 36 NUP214 exons, only 6 different NUP214 exons (23, 28, 29, 31, 32, 34) have been demonstrated to be involved in NUP214-ABL1 rearrangements thus far.^{8,9} In all instances, the coiled-coil domain of NUP214 and the C-terminus involving the SH2, SH3, and tyrosine kinase domains of ABL1 have been consistently implicated in the generation of the NUP214-ABL1 kinase, with molecular weights ranging from 239 kDa to 333 kDa.^{8,9} NUP214 was first described fused with DEK in patients with AML and the t(6; 9)(p23; q34) translocation,²⁰⁻²³ and later with the SET gene.²⁴ NUP214 is a phenylalanine-glycine (FG)-containing cytoplasmic-oriented nuclear pore complex protein implicated in nucleocytoplasmic transport.^{25,26}

N-[2-Chloro-6-methylphenyl]-2-[6-[4-[2-hydroxyethyl]piperazin-1-yl]-2-methylpyrimidin-4-ylamino] thiazole-5-carboxamide; formerly N-(2-chloro-6-methylphenyl)-2-[[6-[4-(2-hydroxyethyl)-1-piperazinyl]-2-methyl-4-pyrimidinyl]amino]-5-thiazolecarboxamide, also known as dasatinib, is a potent, orally available, multi-targeted protein tyrosine kinase inhibitor. Wherever the term “dasatinib” or “N-(2-chloro-6-methylphenyl)-2-[[6-[4-(2-hydroxyethyl)-1-piperazinyl]-2-methyl-4-pyrimidinyl]amino]-5-thiazolecarboxamide” is used herein, it is understood (unless otherwise indicated) that the compound N-(2-chloro-6-methylphenyl)-2-[[6-[4-(2-hydroxyethyl)-1-piperazinyl]-2-methyl-4-pyrimidinyl]amino]-5-thiazolecarboxamide having the following structure (I):

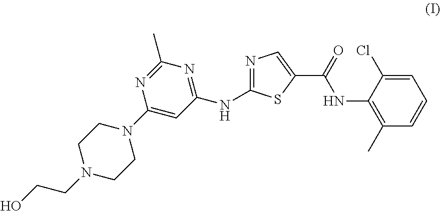

is intended, as well as all pharmaceutically acceptable salts thereof. Compound (I) is also referred to as N-(2-chloro-6-methylphenyl)-2-(6-(4-(2-hydroxyethyl)-1-piperazinyl)-2-methyl-4-pyrimidinyl)amino)-1,3-thiazole-5-carboxamide in accordance with IUPAC nomenclature. Use of the term encompasses (unless otherwise indicated) solvates (including hydrates) and polymorphic forms of the compound (I) or its salts (such as the monohydrate form of (I) described in U.S. patent application Ser. No. 11/051,208, filed Feb. 4, 2005, published as U.S. 2005/0215795 on Sep. 29, 2005, incorporated herein by reference). Pharmaceutical compositions of dasatinib include all pharmaceutically acceptable compositions comprising dasatinib and one or more diluents, vehicles and/or excipients, such as those compositions described in U.S. patent application Ser. No. 11/402,502, filed Apr. 12, 2006, published as U.S. 2006/0235006 on Oct. 19, 2006, incorporated herein by reference. The synthesis and biochemical properties of this compound have been presented previously.⁹ One example of a pharmaceutical composition comprising N-(2-chloro-6-methylphenyl)-2-[[6-[4-(2-hydroxyethyl)-1-piperazinyl]-2-methyl-4-pyrimidinyl]amino]-5-thiazolecarboxamide is SPRYCEL™ (Bristol-Myers Squibb Company). SPRYCEL™ comprises N-(2-chloro-6-methylphenyl)-2-[[6-[4-(2-hydroxyethyl)-1-piperazinyl]-2-methyl-4-pyrimidinyl]amino]-5-thiazolecarboxamide as the active ingredient, also referred to as dasatinib, and as inactive ingredients or excipients, lactose monohydrate, microcrystalline cellulose, croscarmellose sodium, hydroxypropyl cellulose, and magnesium stearate in a tablet comprising hypromellose, titanium dioxide, and polyethylene glycol.

Structural studies indicate that protein tyrosine kinase inhibitors, including dasatinib, bind to the ATP-binding site in ABL, but without regard for the position of the active loop, which can be in the active or inactive conformation.¹⁹ The structure and use of dasatinib as an anticancer agent is described in Lombardo, L. J., et al., J. Med. Chem., 47:6658-6661 (2004) and is described in U.S. Pat. Nos. 6,596,746, granted Jul. 22, 2003 and 7,125,875, granted Oct. 24, 2006, all of which are incorporated by reference herein in their entirety.

Methods for treating an individual suffering from a NUP214-ABL1 positive T cell malignancy can comprise the steps of determining whether a biological sample obtained from the individual comprises NUP214-ABL1, and administering a therapeutically effective amount of dasatanib, nilotinib, or a combination of one or more of dasatinib, nilotinib, and another therapy for treating a NUP214-ABL1 positive T cell malignancy to the individual. Currently, the recommended dosage for dasatinib is twice daily as a 70 mg tablet, or once daily as a 100 mg tablet, referred to as SPRYCEL™. Alternatively, the drug can be administered in combination with a second therapy for treating a NUP214-ABL1 positive T cell malignancy. The second therapy can be any therapy effective in treating a NUP214-ABL1 positive T cell malignancy, including, for example, therapy with another protein kinase inhibitor such as imatinib, AMN107, PD180970, GGP76030, AP23464, SKI 606, NS-187, and/or AZD0530; therapy with a tubulin stabilizing agent for example, pacitaxol, epothilone, taxane, and the like; therapy with an ATP non-competitive inhibitor such as ONO12380; therapy with an Aurora kinase inhibitor such as VX-680; therapy with a p38 MAP kinase inhibitor such as BIRB-796; or therapy with a farnysyl transferase inhibitor. The dosage of dasatinib or nilotinib can remain the same, be reduced, or be increased when combined with a second therapy.

The methods of treating a NUP214-ABL1 positive T cell malignancy in an individual suffering from cancer, will ideally inhibit proliferation of cancerous cells and/or induce apoptosis of the cancerous cells.

Combination treatments comprising a combination of dasatinib and imatinib are described in U.S. Ser. No. 10/886,955, filed Jul. 8, 2004, published as U.S. 2005/0009891 on Jan. 13, 2005; U.S. Ser. No. 11/265,843, filed Nov. 3, 2005, published as U.S. 2006/0094728 on May 4, 2006; and U.S. Ser. No. 11/418,338, filed May 4, 2006, published as 2006/0251723 on Nov. 9, 2006 each of which are incorporated herein by reference in their entirety and for all purposes.

The present invention also provides methods for treating an individual suffering from a NUP214-ABL1 positive T cell malignancy and a BCR-ABL associated disorder, by administering a therapeutically effective amount of dasatinib, nilotinib, or a combination of one or more of dasatinib, nilotinib, and another therapy for treating a NUP214-ABL1 positive T cell malignancy or a BCR-ABL associated disorder.

The term “BCR-ABL” as used herein is inclusive of both wild-type and mutant BCR-ABL.

“BCR-ABL associated disorders” are those disorders which result from BCR-ABL activity, including mutant BCR-ABL activity, and/or which are alleviated by the inhibition of BCR-ABL, including mutant BCR-ABL, expression and/or activity. A reciprocal translocation between chromosomes 9 and 22 produces the oncogenic BCR-ABL fusion protein. The phrase “BCR-ABL associated disorders” is inclusive of “mutant BCR-ABL associated disorders.”

Example disorders include, for example, leukemias, including, for example, chronic myeloid leukemia, acute lymphoblastic leukemia, and Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various additional cancers are also included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain embodiments, the disorder is leukemia, breast cancer, prostate cancer, lung cancer, colon cancer, melanoma, or solid tumors. In certain embodiments, the leukemia is T-ALL, chronic myeloid leukemia (CML), Ph+ ALL, AML, imatinib-resistant CML, imatinib-intolerant CML, accelerated CML, lymphoid blast phase CML.

A “solid tumor” includes, for example, sarcoma, melanoma, carcinoma, or other solid tumor cancer.

The terms “cancer,” “cancerous,” or “malignant” refer to or describe the physiological condition in mammals that is typically characterized by unregulated cell growth. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ ALL).

A “mutant BCR-ABL” encompasses a BCR-ABL tyrosine kinase with an amino acid sequence that differs from wild type BCR-ABL tyrosine kinase by one or more amino acid substitutions, additions or deletions. Wild-type and variant sequences can also be found in the GenBank database. See for example, accession number gi|177943 (encoded by gi|177942), NP_{—}005148.1, and NP_{—}005148.2 (SEQ ID NO. 1).

“Mutant BCR-ABL associated disorder” is used to describe a BCR-ABL associated disorder in which the cells involved in said disorder are or become resistant to treatment with a kinase inhibitor used to treat said disorder as a result of a mutation in BCR-ABL. For example, a kinase inhibitor compound can be used to treat a cancerous condition, which compound inhibits the activity of wild type BCR-ABL which will inhibit proliferation and/or induce apoptosis of cancerous cells. Over time, a mutation can be introduced into the gene encoding BCR-ABL kinase, which can alter the amino acid sequence of the BCR-ABL kinase and cause the cancer cells to become resistant, or at least partially resistant, to treatment with the compound. Alternatively, a mutation can already be present within the gene encoding BCR-ABL kinase, either genetically or as a consequence of an oncogenic event, independent of treatment with a protein tyrosine kinase inhibitor, which can be one factor resulting in these cells propensity to differentiate into a cancerous or proliferative state, and also result in these cells being less sensitive to treatment with a protein tyrosine kinase inhibitor. Such situations are expected to result, either directly or indirectly, in a “mutant BCR-ABL kinase associated disorder” and treatment of such condition will require a compound that is at least partially effective against the mutant BCR-ABL, preferably against both wild type BCR-ABL and the mutant BCR-ABL. In the instance where an individual develops at least partial resistance to the kinase inhibitor imatinib, the mutant BCR-ABL associated disorder is one that results from an imatinib-resistant BCR-ABL mutation, or a protein tyrosine kinase inhibitor resistant BCR-ABL mutation. Similarly, in the instance where an individual develops at least partial resistance to the kinase inhibitor N-(2-chloro-6-methylphenyl)-2-[[6-[4-(2-hydroxyethyl)-1-piperazinyl]-2-methyl-4-pyrimidinyl]amino]-5-thiazolecarboxamide, the mutant BCR-ABL associated disorder is one that results from an N-(2-chloro-6-methylphenyl)-2-[[6-[4-(2-hydroxyethyl)-1-piperazinyl]-2-methyl-4-pyrimidinyl]amino]-5-thiazolecarboxamide resistant BCR-ABL mutation, or a protein tyrosine kinase inhibitor resistant BCR-ABL mutation.

“Imatinib-resistant BCR-ABL mutation” refers to a specific mutation in the amino acid sequence of BCR-ABL that confers upon cells that express said mutation resistance to treatment with imatinib. Mutations that may render a BCR-ABL protein at least partially imatinib resistant can include, for example, E279K, F359C, F359I, L364I, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, I314V, T315I, E316G, F317L, M318T, Y320C, Y320H, G321E, D325H, Y326C, L327P, R328K, E329V, Q333L, A337V, V339G, L342E, M343V, M343T, A344T, A344V, 1347V, A350T, M351T, E352A, E352K, E355G, K357E, N358D, N358S, F359V, F359C, F359I, I360K, I360T, L364H, L364I, E373K, N374D, K378R, V379I, A380T, A380V, D381G, F382L, L387M, M388L, T389S, T392A, T394A, A395G, H396K, H396R, A399G, P402T, T406A, S417Y, and F486S (see, for example, U.S. Publication No. 2003/0158105, incorporated herein by reference in its entirety and for all purposes).

“N-(2-chloro-6-methylphenyl)-2-[[6-[4-(2-hydroxyethyl)-1-piperazinyl]-2-methyl-4-pyrimidinyl]amino]-5-thiazolecarboxamide-resistant BCR-ABL mutation” refers to a specific mutation in the amino acid sequence of BCR-ABL that confers upon cells that express said mutation resistance to treatment with N-(2-chloro-6-methylphenyl)-2-[[6-[4-(2-hydroxyethyl)-1-piperazinyl]-2-methyl-4-pyrimidinyl]amino]-5-thiazolecarboxamide. Such mutations can include the F317I and T315A mutations. Additional mutations that render a BCR-ABL protein at least partially N-(2-chloro-6-methylphenyl)-2-[[6-[4-(2-hydroxyethyl)-1-piperazinyl]-2-methyl-4-pyrimidinyl]amino]-5-thiazolecarboxamide resistant include, for example, T315I. Other mutations are disclosed in PCT Publication No. WO2007/011765, filed Jul. 13, 2006; PCT Publication No. WO2007/065124, filed Nov. 30, 2006; PCT Publication No. WO2007/056177, filed Nov. 3, 2006; and PCT Publication No. WO2007/109527, filed Mar. 16, 2007, and are hereby incorporated by reference in their entirety and for all purposes.

“Imatinib-resistant CML” refers to a CML in which the cells involved in CML are resistant to treatment with imatinib. Generally it is a result of a mutation in BCR-ABL.

“Imatinib-intolerant CML” refers to a CML in which the individual having the CML is intolerant to treatment with imatinib, i.e., the toxic and/or detrimental side effects of imatinib outweigh any therapeutically beneficial effects.

Treatment regimens can be established based upon the detection of NUP214-ABL1. For example, the present invention encompasses screening cells from an individual who may suffer from, or is suffering from, a T cell malignancy. The cells of an individual are screened, using methods known in the art, for identification of NUP214-ABL1. For example, cells may be screened using a reverse-transcriptase polymerase chain reaction (RT-PCR), by performing fluorescence in situ hybridization (FISH), or by any other method known by one skilled in the art.

If NUP214-ABL1 is found in the cells from an individual, treatment regimens can be developed appropriately. For example, the presence of NUP214-ABL1 can indicate that the patient may be responsive to treatment with dasatinib, nilotinib, or a combination of one or more of dasatinib, nilotinib, and another therapy for treating a NUP214-ABL1 positive T cell malignancy.

In addition, treatment regimens can be established based upon the detection of NUP214-ABL1 and a BCR-ABL mutation in the same patient.

A therapeutically effective amount of dasatinib, nilotinib, or a combination of one or more of dasatinib, nilotinib, and another therapy for treating a NUP214-ABL1 positive T cell malignancy, can be orally administered as an acid salt. The actual dosage employed can be varied depending upon the requirements of the patient and the severity of the condition being treated. Determination of the proper dosage for a particular situation is within the skill of the art. The effective amount of dasatinib, nilotinib, or a combination of one or more of dasatinib, nilotinib, and another therapy for treating a NUP214-ABL1 positive T cell malignancy can be determined by one of ordinary skill in the art, and includes exemplary dosage amounts for an adult human of from about 0.05 to about 100 mg/kg of body weight of dasatinib, nilotinib, or a combination of one or more of dasatinib, nilotinib, and another therapy for treating a NUP214-ABL1 positive T cell malignancy, per day, which can be administered in a single dose or in the form of individual divided doses, such as from 1, 2, 3, or 4 times per day. In certain embodiments, dasatinib, nilotinib, or a combination of one or more of dasatinib, nilotinib, and another therapy for treating a NUP214-ABL1 positive T cell malignancy, is administered 2 times per day at 70 mg. Alternatively, it can be dosed at, for example, 50, 70, 90, 100, 110, or 120 BID, or 100, 140, or 180 once daily. It will be understood that the specific dose level and frequency of dosing for any particular subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, and the like, subject to protein tyrosine kinase-associated disorders.

A treatment regimen is a course of therapy administered to an individual suffering from a T cell malignancy that can include treatment with dasatinib, nilotinib, as well as other therapies such as radiation and/or other agents (i.e., combination therapy). When more than one therapy is administered, the therapies can be administered concurrently or consecutively (for example, more than one kinase inhibitor can be administered together or at different times, on a different schedule). Administration of more than one therapy can be at different times (i.e., consecutively) and still be part of the same treatment regimen. Additionally, the combination can be administered with radiation or other known treatments.

Treatment regimens for patients who have both NUP214-ABL1 and a BCR-ABL mutation are also provided herein. In addition to administering a therapeutically effective amount of dasatinib, nilotinib, or a combination of one or more of dasatinib, nilotinib, and another therapy for treating a NUP214-ABL1 positive T cell malignancy to the individual, patients with a BCR-ABL mutation may also be administered a therapeutically effective amount of a BCR-ABL inhibitor. As used herein, a BCR-ABL inhibitor refers to any molecule or compound that can partially inhibit BCR-ABL or mutant BCR-ABL activity or expression. These include inhibitors of the Src family kinases such as BCR/ABL, ABL, c-Src, SRC/ABL, and other forms including, but not limited to, JAK, FAK, FPS, CSK, SYK, and BTK. A series of inhibitors, based on the 2-phenylaminopyrimidine class of pharmacophotes, has been identified that have exceptionally high affinity and specificity for Abl.³⁴ All of these inhibitors are encompassed within the term a BCR-ABL inhibitor. Imatinib, one of these inhibitors, also known as STI-571 (formerly referred to as Novartis test compound CGP 57148 and also known as Gleevec®), has been successfully tested in clinical trail a therapeutic agent for CML. AMN107, is another BCR-ABL kinase inhibitor that was designed to fit into the ATP-binding site of the BCR-ABL protein with higher affinity than imatinib. In addition to being more potent than imatinib (IC50<30 nM) against wild-type BCR-ABL, AMN107 is also significantly active against 32/33 imatinib-resistant BCR-ABL mutants. SKI-606, NS-187, AZD0530, PD180970, CGP76030, and AP23464 are all examples of kinase inhibitors that can be used in the present invention. SKI-606 is a 4-anilino-3-quinolinecarbonitrile inhibitor of Abl that has demonstrated potent antiproliferative activity against CML cell.³⁵ AZD0530 is a dual Abl/Src kinase inhibitor that is in ongoing clinical trials for the treatment of solid tumors and leukemia.³⁶ PD180970 is a pyrido[2,3-d]pyrimidine derivative that has been shown to inhibit BCR-ABL and induce apoptosis in BCR-ABL expressing leukemic cells.³⁷ CGP76030 is dual-specific Src and Abl kinase inhibitor shown to inhibit the growth and survival of cells expressing imatinib-resistant BCR-ABL kinases.³⁸ AP23464 is an ATP-based kinase inhibitor that has been shown to inhibit imatinib-resistant BCR-ABL mutants.³⁹ NS-187 is a selective dual Bcr-Abl/Lyn tyrosine kinase inhibitor that has been shown to inhibit imatinib-resistant BCR-ABL mutants.⁴⁰

A “farnysyl transferase inhibitor” can be any compound or molecule that inhibits farnysyl transferase. The farnysyl transferase inhibitor can have formula (III), (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt. The compound of formula (III) is a cytotoxic FT inhibitor which is known to kill non-proliferating cancer cells preferentially. The compound of formula (III) can further be useful in killing stem cells.

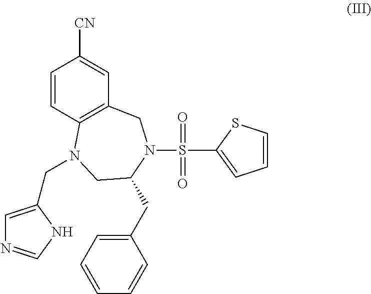

The compound of formula (III), its preparation, and uses thereof are described in U.S. Pat. No. 6,011,029, which is herein incorporated by reference in its entirety and for all purposes. Uses of the compound of formula (III) are also described in WO2004/015130, published Feb. 19, 2004, which is herein incorporated by reference in its entirety and for all purposes.

In practicing the many aspects of the invention herein, biological samples can be selected from many sources such as tissue biopsy (including cell sample or cells cultured therefrom; biopsy of bone marrow or solid tissue, for example cells from a solid tumor), blood, blood cells (red blood cells or white blood cells), serum, plasma, lymph, ascetic fluid, cystic fluid, urine, sputum, stool, saliva, bronchial aspirate, CSF or hair. Cells from a sample can be used, or a lysate of a cell sample can be used. In certain embodiments, the biological sample is a tissue biopsy cell sample or cells cultured therefrom, for example, cells removed from a solid tumor or a lysate of the cell sample. In certain embodiments, the biological sample comprises blood cells.

Pharmaceutical compositions for use in the present invention can include compositions comprising one of dasatinib, nilotinib, or a combination of one or more of dasatinib, nilotinib, and another therapy for treating a NUP214-ABL1 positive T cell malignancy. The determination of an effective dose of a pharmaceutical composition of the invention is well within the capability of those skilled in the art. A therapeutically effective dose refers to that amount of active ingredient which ameliorates the symptoms or condition. Therapeutic efficacy and toxicity can be determined by standard pharmaceutical procedures in cell cultures or experimental animals, for example the ED50 (the dose therapeutically effective in 50% of the population) and LD50 (the dose lethal to 50% of the population).

Dosage regimens involving dasatinib useful in practicing the present invention are described in U.S. Pat. No. 7,125,875; and Blood (ASH Annual Meeting Abstracts) 2004, Volume 104: Abstract 20, “Hematologic and Cytogenetic Responses in imatinib-Resistant Accelerated and Blast Phase Chronic Myeloid Leukemia (CML) Patients Treated with the Dual SRC/ABL Kinase Inhibitor N-(2-chloro-6-methylphenyl)-2-[[6-[4-(2-hydroxyethyl)-1-piperazinyl]-2-methyl-4-pyrimidinyl]amino]-5-thiazolecarboxamide: Results from a Phase I Dose Escalation Study,” by Moshe Talpaz, et al; which are hereby incorporated herein by reference in their entirety and for all purposes.

According to the present invention, dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. See, e.g., the latest Remington's (Remington's Pharmaceutical Science, Mack Publishing Company, Easton, Pa.).

It is to be understood that this invention is not limited to particular methods, reagents, compounds, compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

### Kits

For use in the diagnostic and therapeutic applications described or suggested above, kits are also provided by the invention. Such kits can, for example, comprise a carrier means being compartmentalized to receive in close confinement one or more container means such as vials, tubes, and the like, each of the container means comprising one of the separate elements to be used in the method. For example, one of the container means can comprise a means for detecting whether an individual harbors NUP214-ABL1. Such means can be, for example, RT-PCR or FISH.

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and can also indicate directions for either in vivo or in vitro use, such as those described above.

Kits useful in practicing therapeutic methods disclosed herein can also contain a pharmaceutical composition of dasatinib, nilotinib, or a combination of one or more of dasatinib, nilotinib, and another therapy for treating a NUP214-ABL1 positive T cell malignancy.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

The kit can also comprise, for example, a means for obtaining a biological sample from an individual. Means for obtaining biological samples from individuals are well known in the art, e.g., catheters, syringes, and the like, and are not discussed herein in detail.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The following representative examples contain important additional information, exemplification and guidance which can be adapted to the practice of this invention in its various embodiments and the equivalents thereof. These examples are intended to help illustrate the invention, and are not intended to, nor should they be construed to, limit its scope.

### EXAMPLES

### Example 1

### Methods for Detecting the Presence of NUP214-ABL1 Oncogene

There are several methods for detecting the presence of NUP214-ABL1 in cancer patients, particularly T-ALL patients. They include, but are not limited to, screening a biological sample from an individual for NUP214-ABL1 using a reverse transcription reaction and/or fluorescence in situ hybridization (FISH).

The frequency of the NUP214-ABL1 oncogene among a group of adult patients with T cell malignancies was investigated using reverse transcriptase-polymerase chain reaction and fluorescence in situ hybridization (FISH). Experiments were performed as follows:

### Clinical Samples

Bone marrow (BM) and peripheral blood (PB) samples at diagnosis were available from 29 of 129 patients with T-cell malignancies registered in the Department of Leukemia tissue bank at M.D. Anderson Cancer Center, Houston, Tex. (MDACC). Mononuclear cells from PB or BM samples were separated by Histopaque (density 1.077) gradient centrifugation. Contaminating red cells were lysed in 0.8% ammonium chloride solution (StemCell Technologies, Vancouver, Canada) for 10 minutes. The research protocol was approved by the Institutional Review Board at MDACC.

### Reverse Transcriptase-Polymerase Chain Reaction (RT-PCR) Analysis of NUP214-ABL1 Transcripts

RNA was extracted using TRIzol reagents (Invitrogen, Carlsbad, Calif.) following manufacturer's recommendations. RNA was reverse transcribed into cDNA using random hexamer and reverse transcriptase with the SuperScript First-Strand Synthesis System kit for RT-PCR (Invitrogen, Carlsbad, Calif.) according to the manufacturer's protocol. After reverse transcription, cDNA was amplified using the primers NUP23 (exon 23), NUP29 (exon 29), NUP31 (exon 31) and NUP32 (exon 32), NUP34 (exon 34), combined with ABL1-R1 primer. The primer sequences were as follows:

| | |
|---|---|
| NUP23 (5-TAGTCCTTCCCACCCCATCT-3), | (SEQ ID NO: 2) |
| NUP29 (5-AGGGAGGCTCTGTCTTTGGT-3), | (SEQ ID NO: 3) |
| NUP31 (5-TTGGAGGAAAACCCAGTCAG-3), | (SEQ ID NO: 4) |
| NUP32 (5-GCTCTGGAGGAGGAAGTGTG-3), | (SEQ ID NO: 5) |
| NUP34 (5-TGGTTTTGGGACCCAGAGTA-3) | (SEQ ID NO: 6) |
| and | |
| ABL1-R1 (5-GGTTGGGGTCATTTTCACTG-3). | (SEQ ID NO: 7) |

PCR was carried at 94° C. for 30 seconds, 58° C. for 30 seconds, and 72° C. for 30 seconds for 45 cycles. The final PCR products were separated on a 1% agarose gel with ethidium bromide and visualized under ultraviolet light. The appropriate bands were cut and purified using Qiaquick gel extraction kit (Qiagen, Valencia, Calif.). Purified PCR products were ligated into PCR2.1-TOPO cloning vector and transformed into One Shot TOP10 competent cells using TOPO TA Cloning kit (Invitrogen, Carlsbad, Calif.). Clones were then sequenced using standard techniques on an ABI sequencer using M13 primers. The primer sequences used for nested PCR were as follows: NUP214-31F2 (5-CCAACAAAAACCCATTCAGC-3) (SEQ ID NO: 8) and NUP214-31R2 (5-GTTGGGGTCATTTTCACTGG-3) (SEQ ID NO: 9).

### Fluorescence In Situ Hybridization (FISH)

To detect NUP214-ABL1 by FISH, one bacterial artificial chromosome (BAC) clone which overlaps NUP214 (RP11-544A12; 132,960-133,150 KB on chromosome 9q) and one clone that overlaps ABL1 (RP11-83J21; 132,640-132,820 KB on chromosome 9q) were selected using mapping information from the National Center of Biotechnology Information (NCBI). Briefly, these BAC clones were grown in TB media with 20 μg/ml chloramphenicol. DNA was isolated using a standard alkaline lysis kit (Eppendorf Plasmid Mini Prep). DNA extracted from one BAC clone was labeled using digoxigenin-11-UTP or biotin-UTP by nick translation and detected with anti-digoxigenin-rhodamine (red) or avidin-FITC (green) fragments. Thus, we produced a NUP214-ABL1 red/green fusion FISH probe. Metaphase chromosome spreads, for the performance of FISH analysis to verify that the fluorescently labeled BAC clones hybridized to the correct chromosome location on 9q, were obtained using standard procedures. Patient samples (BM or PB smears), to which the NUP214-ABL1 fusion FISH probes were hybridized, were analyzed using a Zeiss Axiphot fluorescent microscope including single- and triple-band pass filters. Digital FISH images were captured by a Power Macintosh G3 System and MacProbe version 4.4 (Applied Imaging, San Jose, Calif.).

### Results

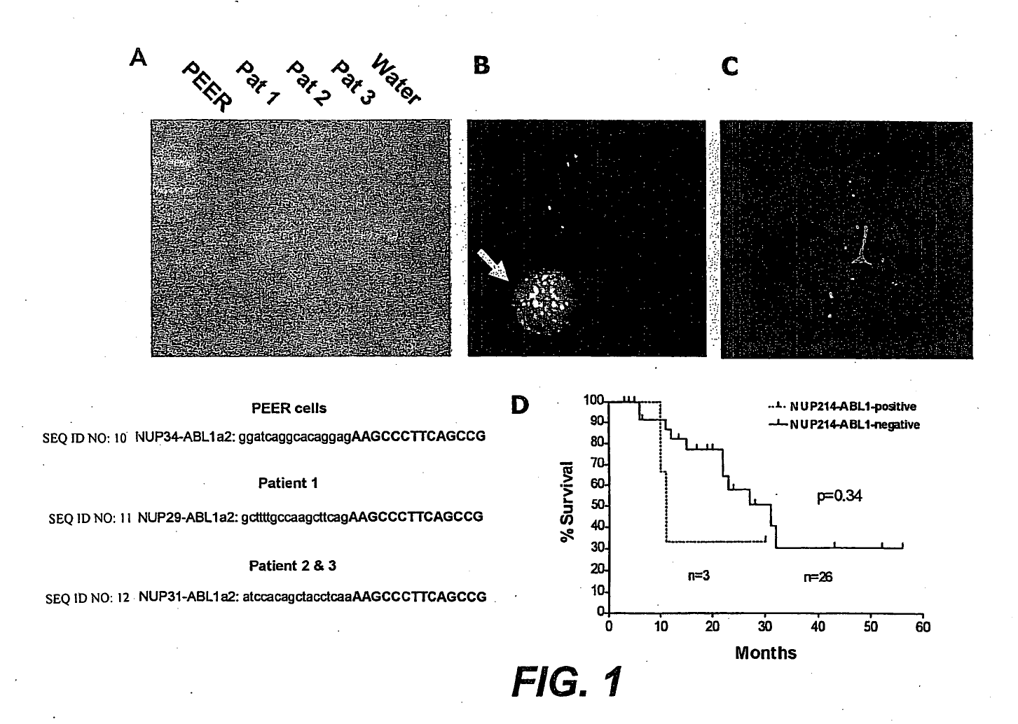

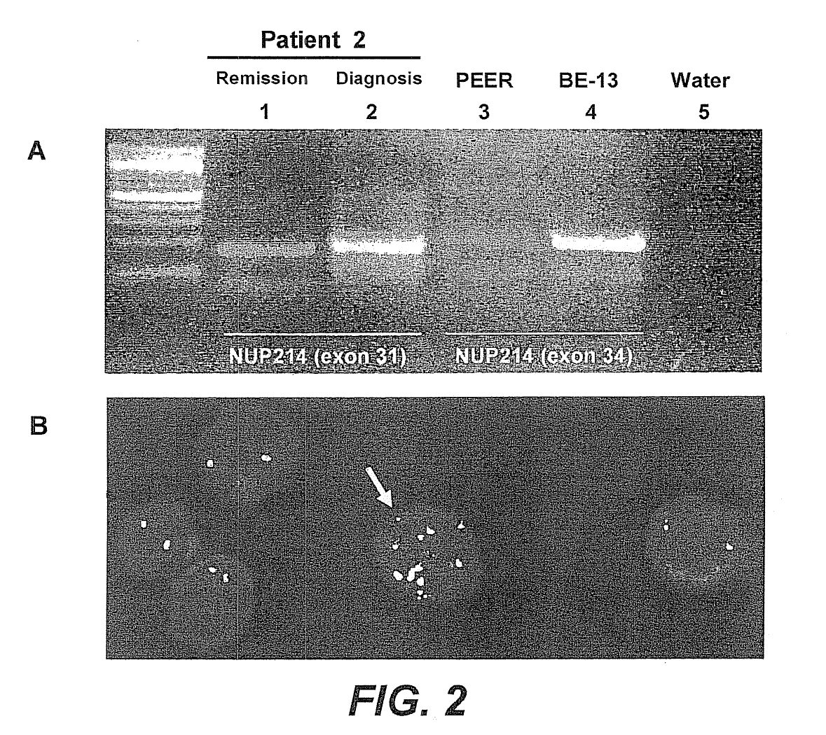

29 patients with T cell malignancies (23 with T-ALL and 6 with T-lymphoblastic lymphoma) were screened for the presence of NUP214-ABL1 rearrangements using specific primers against NUP214 exons 23, 29, 31, 32, and 34, and ABL1 exon a2. NUP214-ABL1 transcripts were demonstrated in 3 (10%) patients by RT-PCR. The sequences of the encountered transcripts demonstrate the involvement of in-frame fusions between exon a2 of ABL1 and exon 29 (in Patient 1) and exon 31 (in Patients 2 & 3) of NUP214 (FIG. 1A). This was confirmed by direct sequencing in all cases. The presence of NUP214-ABL1 was also investigated using a panel of BAC clones overlapping NUP214 or ABU, both on chromosome 9q34. Cohybridization of the 3 ABL1 and NUP214 probes (more than 12 signals per nucleus) confirmed the presence of overamplification of the NUP214-ABL1 fusion oncogene by FISH analysis in all 3 NUP214-ABL1-positive patients by RT-PCR (FIGS. 1B and 1C).

The characteristics of the 3 NUP214-ABL1-positive patients are shown in Table 1.

TABLE 1
Clinical features of the NUP214-ABL1-positive patients with T cell malignancies

| Patient | | | | | | % PB | % BM | Extramedullary | |
|---|---|---|---|---|---|---|---|---|---|
| No. | Sex | Age | Material | Diagnosis | WBC | blast | blast | involvement | Phenotype |
| 1 | F | 37 | BM | T-ALL | 13.1 | 72 | 49 | Skin | Precursor |
| | | | | | | | | | T cell |
| 2 | F | 40 | PB & | T-LL | 6.6 | 9 | 12 | Mediastinal | Precursor |
| | | | BM | | | | | mass | T cell |
| 3 | M | 37 | BM | T-LL | 10.6 | 0 | 0 | Mediastinal | Precursor |
| | | | | | | | | mass | T cell |

| | | NUP214- | Response | | |
|---|---|---|---|---|---|
| Patient | | ABL 1 | to | | |
| No. | Karyotype | transcript | hyperCVAD | Relapse | Status |
| 1 | 46, XX[10]; | NUP29-a2 | CR after 2 | no | Dead |
| | 46 XX, | | cycles | | (PCP) |
| | t(7; 14) | | | | |
| | (p22; q22), | | | | |
| | i(17q) [10] | | | | |
| 2 | 46, XX | NUP31-a2 | CR after 1 | CR by flow | CR, |
| | | | cycle | cytometry | 19+ |
| | | | | | months |
| 3 | 46, XY | NUP31-a2 | CR after 1 | Mediastinal | Dead |
| | | | cycle | and | (DP) |
| | | | | leptomeningeal | |
| | | | | after 6 cycles | |
| | | | | of HyperCVAD | |

Abbreviations: F: female; M: male; T-ALL: T cell acute lymphoblastic leukemia; T-LL: T cell lymphoblastic lymphoma; WBC: peripheral blood leukocyte count (×10⁹/L); CR: complete remission; PCP: Pneumocystis Carinii pneumonia; DP: disease

No significant differences were observed at diagnosis between NUP214-ABL1-positive and -negative patients, except for a lower marrow blast percentage in patients expressing NUP214-ABL1, as 2 of these 3 patients had T-lymphoblastic lymphoma (T-LL). The median age of NUP214-ABL1-positive was 37 years (range, 37 to 40). Patient 1 was diagnosed with precursor T-ALL and presented with leukemia cutis, whereas Patients 2 and 3 presented with a mediastinal mass and were diagnosed with precursor T-LL. All patients received therapy with hyperCVAD (hyper fractionated cyclophosphamide, vincristine, doxorubicin, and dexamethasone alternating with high dose methotrexate and ara-C).¹⁸ Patients 2 and 3 received additional mediastinal radiotherapy. Patient 1 achieved complete remission (CR) after 2 cycles of hyperCVAD but expired due to mediastinal and leptomeningeal relapse after 6 cycles of chemotherapy. Patient 3 expired in CR after 9 cycles of hyperCVAD due to pneumocystis carinii pneumonia. Patient 2 achieved a CR after the first cycle of hyperCVAD, continued this therapy for a total of 8 cycles, and is currently receiving maintenance chemotherapy with oral POMP (mercaptopurine, methotrexate, vincristine, and prednisone). No significant differences were observed regarding overall survival between NUP214-ABL1-positive and -negative patients (p=0.34) (FIG. 1D).

Patient 2, with precursor T-LL, was found to express the NUP214-ABL1 transcript NUP31-a2 by PCR analysis. Immunohistochemistry and flow cytometry assays on PB and BM samples after 9 cycles of POMP maintenance chemotherapy failed to demonstrate any evidence of disease. RNA extracted from PB was reverse transcribed into cDNA. The presence of the NUP214-ABL1 transcript could not be demonstrated upon cDNA amplification using specific primers for the NUP31-a2 rearrangement. However, when PCR was performed using the initial PCR product with nested primers, a conspicuous band with similar size to the pre-treatment NUP214-ABL1 transcript was observed (FIG. 2A). The presence of the NUP31-a2 rearrangement was confirmed by direct sequencing and NUP214-ABL1 was also demonstrated by FISH in a synchronous bone marrow specimen (FIG. 2B).

### Example 2

### Determining the Sensitivity of NUP214-ABL1 Positive Human T-all Cells to Imatinib, Nilotinib, and Dasatinib

### Compounds and Cell Lines

Imatinib and nilotinib were a gift from Dr. Miroslav Beran, M.D. Anderson Cancer Center (MDACC, Houston, Tex.) and dasatinib was provided by Bristol-Myers Squibb Oncology (Princeton, N.J.). All drugs were stored as a 10 mM stock solution in dimethyl sulfoxide (DMSO) and diluted in RPMI 1460 media for use. Aliquots were stored at −20° C. (imatinib and dasatinib) or at 4° C. (nilotinib), respectively.

Antibodies and their sources were as follows: anti-CrKL (32 H4), anti-phospho-CrKL (Tyr207), anti-c-Abl, anti-phospho-c-Abl (Tyr245)(73E5), and anti-phospho-c-Abl (Tyr412)(247C7) antibodies were purchased from Cell Signaling Technology (Beverly, Mass.). Anti-caspase-3 antibody was purchased from eBioscience (San Diego, Calif.). Anti-STAT5A, anti-phospho-STAT5A/B (Tyr694/699), anti-caspase-9, anti-PARP, and anti-Bcl-2 antibodies were obtained from Upstate (Temecula, Calif.). Antibody directed against the C-terminal part of NUP214 was a gift from Dr. Gerard Grosveld (St. Jude Children's Research Hospital, Memphis, Tenn.). Mouse anti-β-Actin monoclonal antibody was from Sigma (St Louis, Mo.), and HPR-linked anti-mouse and anti-rabbit IgG were purchased from Amersham Biosciences (Arlington Heights, Ill.).

Four T-ALL cell lines were used: SIL-ALL, PEER, and BE-13, which carry the NUP214-ABL1 transcript, and Jurkat, which does not express this fusion gene. PEER and Jurkat cell lines were purchased from the American Type Culture Collection (ATCC, Manassas, Va.). SIL-ALL and BE-13 were purchased from the Deutsche Sammlung von Mikroorganismen and Zellkulturen GmbH (DSMZ, Braunschweig, Germany). The cell lines PEER and BE-13 show identical DNA fingerprints, which suggest a common genetic origin. However, PEER features a pseudodiploid karyotype whereas BE-13 is tetraploid, suggesting that BE-13 derives from PEER cells.

All T-ALL cell lines were cultured in RPMI 1640 medium (Invitrogen, Carlsbad, Calif.) supplemented with 10% fetal bovine serum (FBS; Hyclone, Logan, Utah), 100 U/ml penicillin G, and 100 μg/ml streptomycin at 37° C. under 5% CO2. Human bone marrow (BM) cells were cultured in RPMI 1640 medium supplemented with 10% FBS for 3 hours in the presence of nilotinib or dasatinib.

### Cell Proliferation Inhibition Assay

Exponentially growing T-ALL cell lines were plated at 1×10⁴ cells per well in 96-well plates in RPMI 1640 medium supplemented with 10% FCS. The exponentially growing PEER and BE-13 cells were exposed to increasing concentrations of imatinib, nilotinib, and dasatinib up to 10 μM. Viable cell number was assessed 72 hours postplating by the 3 methanethiosulfonate (MTS)-based viability assay (CellTiter 96®Aqueous One Solution Reagent, Promega Corporation, Madison, Wis.) as described.¹⁶ Triplicate assays were averaged, and absorbance at 595 nm (A₅₉₅) versus concentration of imatinib, nilotinib, or dasatinib was graphed as a best-fit sigmoidal curve by using a single-site, nonlinear curve-fitting algorithm (GraphPad Software, San Diego, Calif.).

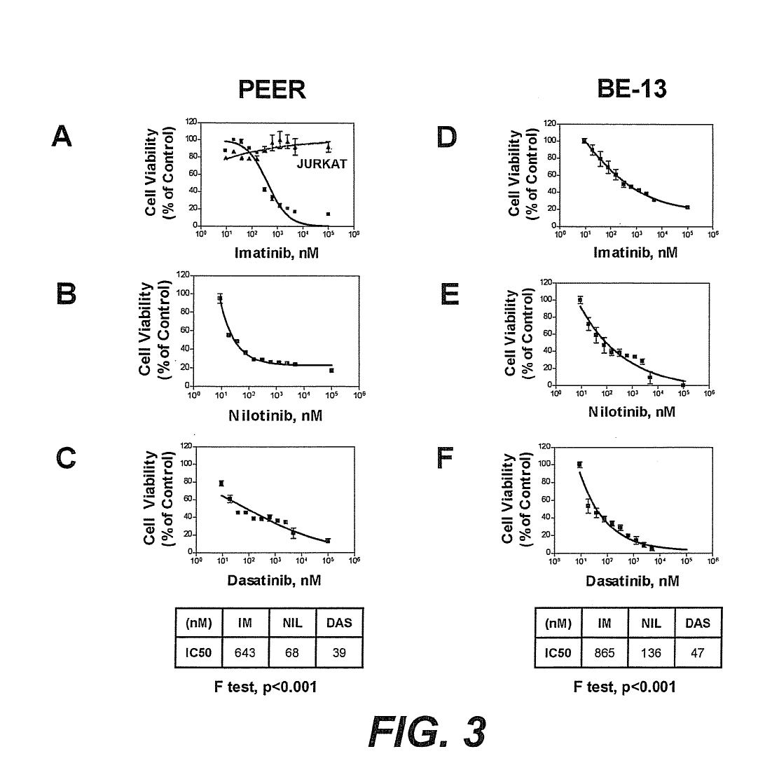

PEER cell viability was significantly reduced after 72 hours of treatment with all 3 TKIs (FIG. 3). However, the IC₅₀ was almost 10-fold higher for imatinib (643 nM) than for nilotinib (68 nM) or dasatinib (IC₅₀ 39 nM) (F test, p<0.001) (FIG. 3A-C). This is consistent with the higher ABL1 kinase inhibitory activity in vitro of nilotinib and dasatinib compared to imatinib in BCR-ABL1-positive cells.^{10,12} BE-13 cells proved slightly less sensitive to imatinib (IC₅₀ 865 nM), nilotinib (IC₅₀ 136 nM), or dasatinib (IC₅₀ 47 nM) than PEER cells (FIG. 3D-F). By contrast, the NUP214-ABL1-negative T-ALL cell line Jurkat, was remarkably resistant to imatinib with IC₅₀ values greater than 10 μM (FIG. 3A), indicating that the cytotoxicity induced by either of these TKIs is not related to a general toxic effect on T-ALL cell lines. Similar results were observed when Jurkat cells were exposed to nilotinib or dasatinib (data not shown). Interestingly, SIL-ALL cells were highly sensitive to dasatinib with IC₅₀ values of 0.65 nM (data not shown).

### Apoptosis Assay

T-ALL cells were incubated in the presence of imatinib, nilotinib, or dasatinib for 24, 48, and 72 hours, pelleted, washed in Ca²⁺-free PBS, and resuspended in 100 μl of annexin V binding buffer (10 mM 4-[2-hydroxyethyl]-1-piperazineethane-sulfonic acid, [pH 7.4]; 0.15 M NaCl; 5 mM KCl; 1 mM MgCl₂; 1.8 mM CaCl₂) before the fluorogenic substrate annexin V-fluoroisothiocyanate (Trevigene, Gaithersburg, Md.) was added to monitor annexin V activity by flow cytometry. Next, cells were incubated for 15 minutes at room temperature in the dark and then washed in 2 ml Ca²⁺-free PBS and resuspended in 0.5 ml of binding buffer. Propidium iodide (PI) was added to allow identification and exclusion of cells that had lost membrane integrity during analysis. Binding of annexin V to apoptotic cells was analyzed with a FACSort flow cytometer (Becton Dickinson Systems, San Jose, Calif.) equipped with Cell Quest Pro software (Becton Dickinson). Data were analyzed using the Mod Fit LT v3.1 software (Verity Software House Inc., Topsham, Me.).

### Cell Cycle Analysis

Exponentially growing T-ALL cells were incubated with TKIs at their IC₂₀ and IC₈₀ concentrations for 24, 48, or 72 hours, pelleted, washed in Ca²⁺-free PBS, and fixed overnight in 70% cold ethanol at −20° C. Next, cells were washed twice in cold PBS, resuspended in hypotonic PI solution (25 μg/ml of propidium iodide, 0.1% Triton X-100, 30 mg/ml of polyethylene glycol, and 3600 units/ml of RNase, dissolved in 4 mM/1 sodium citrate buffer [pH 7.8]; Sigma) and incubated for at least 1 hour at 4° C. in the dark. Cell cycle distribution was analyzed by using a FACSort flow cytometer equipped with ModFit LT v3.1 software (Verity Software House Inc., Topsham, Me.). Cells with hypodiploid DNA were considered apoptotic.

### Western Blot Analysis

Control cells and cells treated with the test TKIs, were rinsed with PBS and then subjected to protein extraction with 1 ml lysis buffer containing 20 mM Tris-HCl, pH 7.4, 10% v/v SDS, 1 mM EDTA, 25 μg/ml aprotinin, and 25 μg/ml pepstatin at 4° C. The DNA in the lysates was sheared by rapidly passing the lysate 10 times through a 23-gauge needle or by sonication. Antibodies were added to aliquots of lysates equalized for protein content by the Bradford assay (Bio-Rad; using the BSA standard). Sodium dodecyl sulfate-polyacrylamide gel electrophoresis (SDS-PAGE) and immunoblotting were described previously¹⁷

### Results

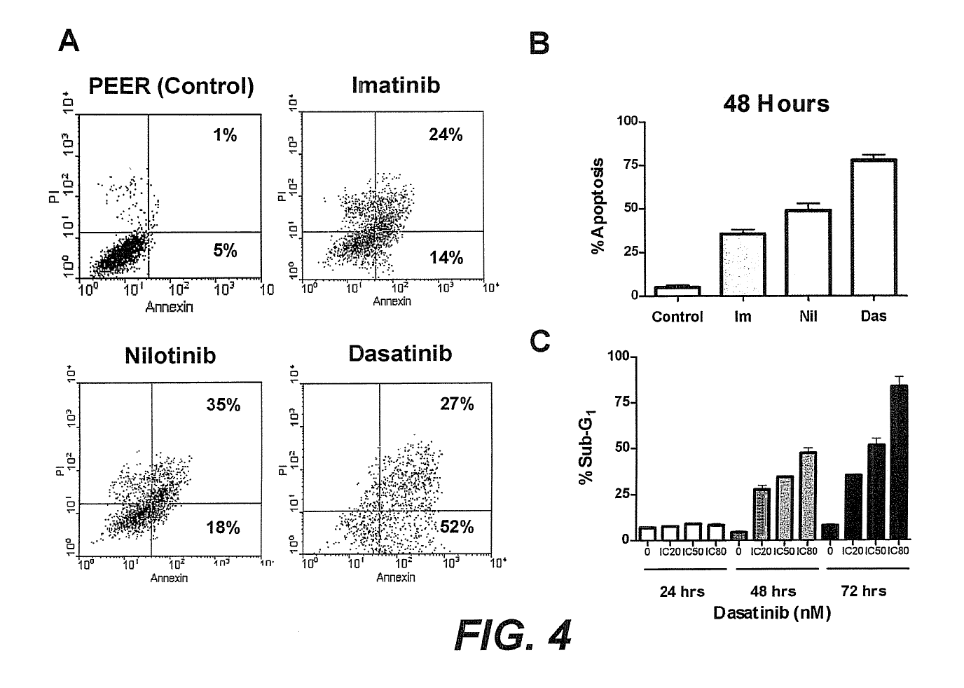

It was analyzed whether the ability of imatinib, nilotinib, and dasatinib to inhibit the growth of NUP214-ABL1-positive cells was associated with induction of apoptosis. Flow cytometry analysis of PEER cells subjected to annexin V/PI double staining revealed that the percentage of annexin V-positive cells increased in a time- and dose-dependent manner after treatment with all 3 TKIs at their respective IC₅₀ concentrations, indicating apoptotic cell death. After exposure for 48 hours, and as expected based on cell proliferation assays, apoptosis was more pronounced in cells treated with dasatinib as compared with those exposed to imatinib or nilotinib (FIGS. 4A and 4B). Exposure of PEER cells to increasing concentrations of dasatinib for 48 hours led to enhanced accumulation of PEER cells in the sub-G1 cell cycle phase, indicating DNA fragmentation and degradation, which is an early event in the apoptotic process (FIG. 4C). Similar data were obtained in BE-13 cells (data not shown).

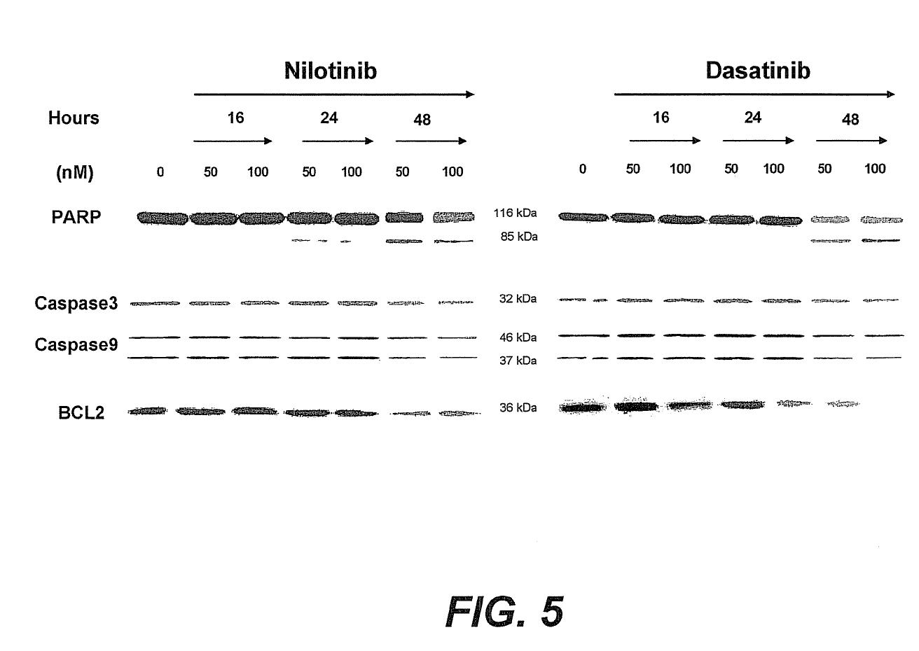

The impact of nilotinib and dasatinib on the expression of apoptosis-related proteins was investigated to further define the apoptotic mode of cell death induced by these TKIs. (FIG. 5). PEER cells were exposed to nilotinib and dasatinib at 50 nM or 100 nM for 16, 24, and 48 hours after seeding. This resulted in significant cleavage of PARP (85 kDa fragment) after 24 hours of treatment. Furthermore, treatment with either TKI led to time-dependent proteolytic activation of caspase-3 and caspase-9, which are responsible for the activation of key proteins involved in the caspase cascade leading to apoptosis. Exposure to both compounds also resulted in diminished levels of the anti-apoptotic protein BCL-2, particularly after 48 hours of treatment with dasatinib 100 nM (FIG. 5).

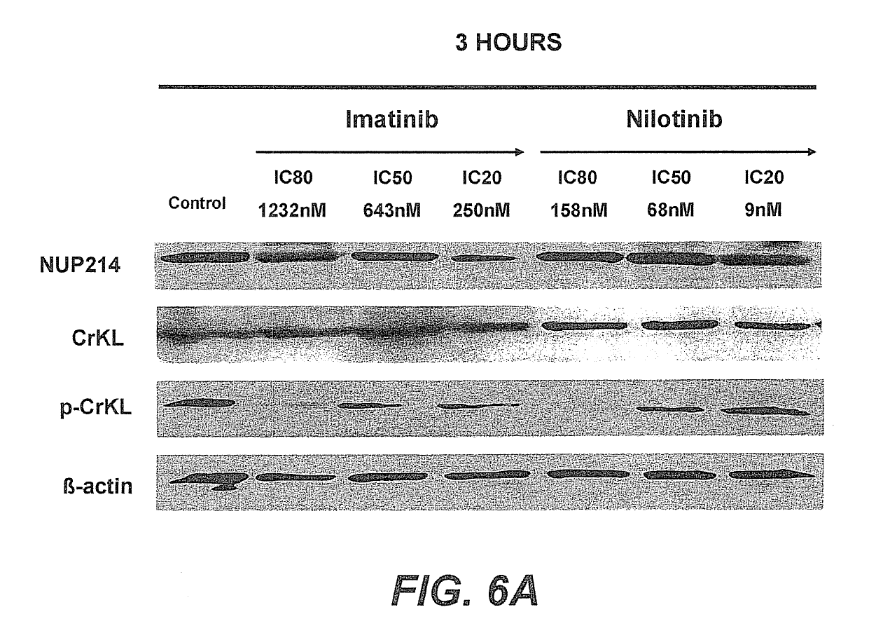

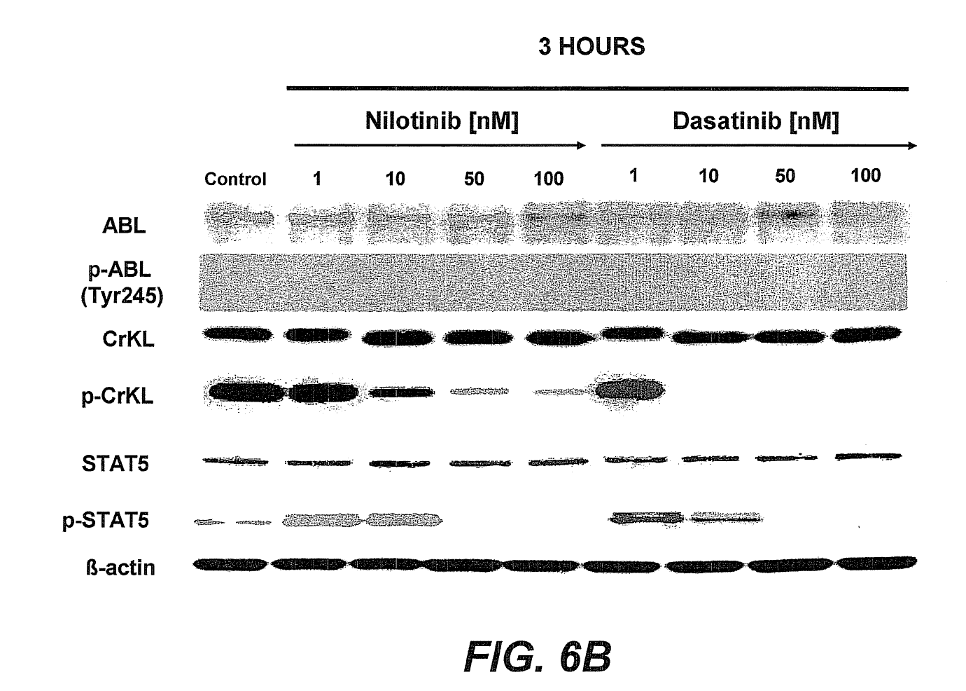

Furthermore, since phosphorylation is critical in NUP214-ABL1 signaling, and this oncogenic kinase is conceivably a target for TKIs, the activity of imatinib and nilotinib against signaling elements downstream NUP214-ABL1 was investigated. To this end, PEER cells were exposed to escalating concentrations of imatinib or nilotinib for 3 hours. Nilotinib is structurally related to imatinib but has 30-fold higher potency against ABL kinase. CrKL is an SH2 (SRC homology domain)/SH3-containing adaptor protein that is a direct target of ABL1 kinase.^{9,19-20}CrKL couples nonreceptor tyrosine kinases to downstream signaling cascades that regulate gene expression²¹ and the specificity of CrKL phosphorylation to BCR-ABL1 signaling supports its acceptance as a surrogate of Abl kinase status.²² Exposure of PEER cells to increasing concentrations of imatinib and nilotinib for 3 hours resulted in inhibition of CrKL phosphorylation, but this was more pronounced upon nilotinib exposure (FIG. 6A). Exposure to escalating concentrations of either TKI did not affect significantly the expression of the NUP214 protein. Altogether, these results suggest that ABL kinase may be a direct target of TKIs in NUP214-ABL1-positive leukemic cells.

Since dasatinib and nilotinib are significantly more potent than imatinib against ABL1 kinase,^{10,12} the inhibitory activity of dasatinib was directly compared with that of nilotinib against NUP214-ABL1-positive cells. Exposure of PEER cells to increasing concentrations of both TKIs (range, 1 nM to 100 nM) for 3 hours led to reduced ABL1 phosphorylation (FIG. 6B). ABL1 phosphorylation was detected with specific antibodies against Tyr245, which is located in the linker region between the SH2 and the catalytic domains. Phosphorylation of this residue is important for the activation of ABL kinase activity.²³ However, phosphorylation at Tyr412 could not be shown (data not shown), mapping to the kinase activation loop of ABL1, whose phosphorylation is also required for ABL1 kinase activity. When the membrane was stripped and reprobed with anti-ABL1 antibody, the amount of total ABL1 remained unchanged, indicating that imatinib and nilotinib abolished ABL1 phosphorylation without altering ABL1 expression (FIG. 6B).

Exposure of PEER cells to nilotinib and dasatinib resulted in dramatic reduction in phosphorylation of CrKL. However, dasatinib concentrations as low as 10 nM resulted in complete abrogation of CrKL phosphorylation, an effect that could not be observed with concentrations of nilotinib up to 100 nM. These findings are in concert with the 1-log higher activity of dasatinib against ABL kinase.¹² In addition, the phosphorylation of STAT5, a downstream target of ABL1 kinase and a recurrent theme in cellular transformation by tyrosine kinase fusion proteins, was also inhibited by both, nilotinib and dasatinib. The inhibition of CrKL and STAT5 phosphorylation proved to be time-dependent. Since the inhibition of phosphorylation of CrKL and STAT5 antedates the induction of apoptosis of PEER cells, it is reasonable to hypothesize that the inhibition of the activation of these molecules may contribute to the proapoptotic effect of these TKIs in NUP214-ABL1-positive cells.

### NUP214-ABL1-Positive Leukemia Xenograft Murine Model

In order to assess whether the in vitro anti-proliferative activity of dasatinib against imatinib-resistant CML cell lines also translated into in vivo efficacy, experiments were performed using mouse xenograft models of imatinib-resistant CML. Briefly, the experiments were performed as follows:

ALL-SIL cells were suspended (2×10⁸ cells/ml) in RPMI 1640 medium. 0.1 mL of the suspension was injected subcutaneously into the ventral axillary region of female NOD/SCID mice (Harlan, Indianapolis). Tumors were staged to a size of 150-300 mg and animals were evenly distributed to various treatment and control groups (8 mice per group). For administration to mice, dasatinib was dissolved in a mixture of propylene glycol/water (50:50). Animals were treated with dasatinib or placebo 0.01 ml/gm of mice every 24 hours by oral gavage. Tumor response was determined twice weekly by measurement of tumors with a caliper until they reached a target size of 1 gm. Tumor weight was estimated from the formula: Tumor weight=(length×width 2)÷2.

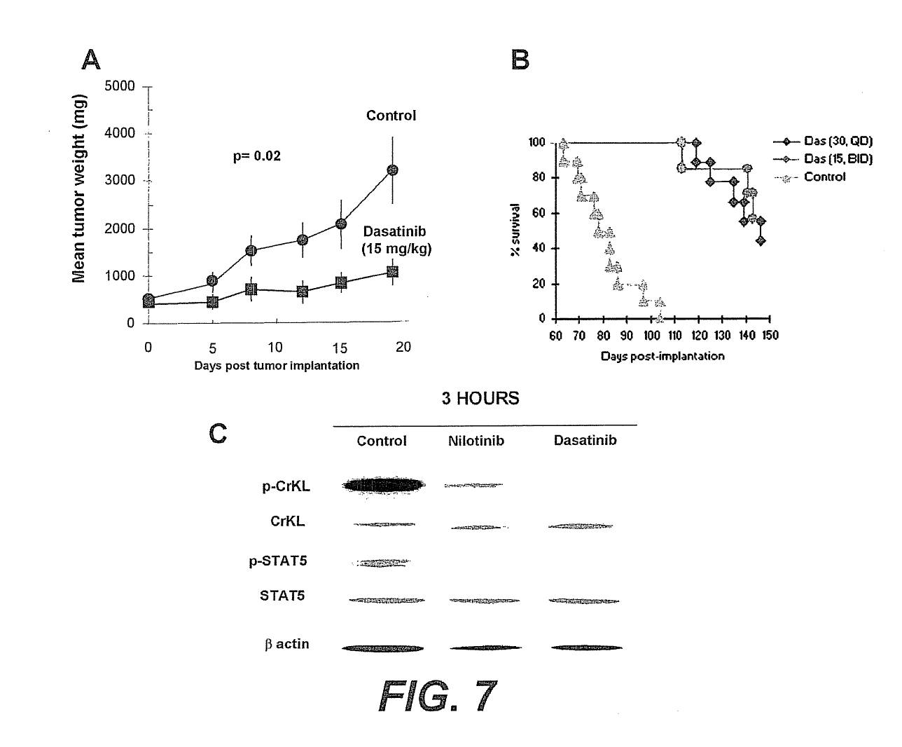

Administration of dasatinib to mice resulted in remarkable growth inhibition of SIL-ALL tumors after 19 days of treatment compared with control animals treated with placebo (1085 mg vs 3236 mg; p=0.02) (FIG. 7A). The efficacy of dasatinib and nilotinib against primary human NUP214-ABL1-positive T-ALL cells was assessed by treating BM leukemic blasts obtained from Patient 1 at diagnosis with both drugs (FIG. 7B). This BM sample had 72% involvement by lymphoblasts with precursor T cell immunophenotype (Table 1). BM blasts were cultured for 3 hours in the presence of dasatinib and nilotinib at their predicted IC₁₀₀ concentrations on PEER cells (180 nM and 220 nM, respectively). As shown in FIG. 7B, treatment with nilotinib resulted in significant (albeit partial) reduction of phosphorylation of CrKL and STAT5, while this was completely abrogated when primary BM cells were treated with an equipotent concentration of dasatinib, recapitulating the results obtained in NUP214-ABL1-positive cell lines. These results indicate that NUP214-ABL1 tyrosine kinase is constitutively activated in vivo, activates downstream signaling elements similar to BCR-ABL1 kinase, and consequently is amenable to inhibition by potent TKIs such as dasatinib and nilotinib.

### Discussion

Extrachromosomal oncogene amplification has been described on double-minute (dmin) chromosomes²⁸ and on certain structures below the threshold of detection of conventional cytogenetics termed episomes.³⁰ Graux et al identified 5 different NUP214-ABL1 transcripts among 85 patients with T-ALL who displayed episomal ABL1 overamplification and demonstrated the selective absence of the 5′ end of ABL1 in the amplicon, in concordance with the involvement of ABL1 in the generation of the fusion gene.⁸ Other NUP214-ABL1 genomic presentations have also been demonstrated, including intrachromosomal amplification and 9q34 insertions, which can coexist in the same patient.¹⁸ Recently, the NUP214-ABL1 fusion has been reported in 11 (3.9%) of 279 patients with T-ALL by means of a multiplex RT-PCR approach that included 10 different NUP214 forward primers and 2 ABL1 primers (a2 and a3) to allow amplification of all possible NUP214-ABL1 in-frame transcripts.⁹ We have found 3 NUP214-ABL1-positive patients among 29 screened. The presence of this fusion transcript was demonstrated by both RT-PCR and FISH, with excellent concordance between both techniques. NUP214-ABL1 is frequently associated with deletion of the tumor suppression genes CDKN2A and CDKN2B (p15)⁸ and overexpression of the transcription factors TLX1^{6,7} (HOX11) or TLX3^{8,9}, 28 (HOX11L2). Although NUP214-ABL1-positive patients, particularly those who additionally overexpress the transcription factor TLX3 (HOX11L2),³¹ had been initially been linked to a poorer outcome,⁸ data from the present study, in which all patients were uniformly treated with hyperCVAD, and from a large cohort of 279 adult patients with T-ALL treated with the German multicenter adult ALL (GMALL) trials,⁹ do not support a difference in overall survival between NUP214-ABL1-positive and -negative patients.

Our experiments show that imatinib, nilotinib, and dasatinib inhibit the growth of PEER and BE-13 cells but nilotinib and dasatinib exhibit >1-log higher antiproliferative activity than imatinib against these cell lines. The remarkable antiproliferative activity of dasatinib was associated with complete abrogation of CrKL phosphorylation, a surrogate marker of ABL1 kinase activity, and STAT5, a molecule whose activation is a recurrent theme in cellular transformation by tyrosine kinase fusion proteins.^{33,34} The latter events antedate the proteolytic activation of caspase-3 and caspase-9, and BCL-2 downregulation, which suggests that abrogation of CrKL and STAT5 activation may be responsible for the proapoptotic effect of TKIs in NUP214-ABL1-positive cells. More important, treatment with dasatinib of NUP214-ABL1-positive T cell lymphoblasts obtained from the BM of one patient with T-ALL led to complete abrogation of CrKL and STAT phosphorylation, recapitulating our in vitro results, which indicates a potential for this agent in the treatment of patients with NUP214-ABL1-positive T cell malignancies.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The entire disclosure of each document cited (including patents, patent applications, journal articles, abstracts, laboratory manuals, books, Genbank Accession numbers, SWISS-PROT Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

### REFERENCES

1. Daley G Q, Van Etten R A, Baltimore D. Induction of chronic myelogenous leukemia in mice by the P210 bcr/abl gene of the Philadelphia chromosome. Science. 1990; 247:824-830.
2. Heisterkamp N, Jenster G, ten Hoeve J, Zovich D, Pattengale P K, Groffen J. Acute leukaemia in bcr/abl transgenic mice. Nature. 1990; 344:251-253.
3. Faderl S, Kantarjian H M, Thomas D A, et al. Outcome of Philadelphia chromosome-positive adult acute lymphoblastic leukemia. Leuk Lymphoma. 2000; 36:263-273.
4. Lugo T G, Pendergast A M, Muller A J, Witte O N. Tyrosine kinase activity and transformation potency of bcr-abl oncogene products. Science. 1990; 247:1079-1082.
5. Colleoni G W, Yamamoto M, Kerbauy J, et al. BCR-ABL rearrangement in adult T-cell acute lymphoblastic leukemia. Am J Hematol 1996; 53:277-8.
6. Fabbiano F, Santoro A, Felice R, Catania P, Cannella S, Majolino I. bcr-abl rearrangement in adult T-lineage acute lymphoblastic leukemia. Haematologica 1998; 83:856-7.
7. Barber K E, Martineau M, Harewood L, et al. Amplification of the ABL gene in T-cell acute lymphoblastic leukemia. Leukemia 2004; 18:1153-6.
8. Graux C, Cools J, Melotte C, et al. Fusion of NUP214 to ABL1 on amplified episomes in T-cell acute lymphoblastic leukemia. Nat Genet. 2004; 36:1084-9.
9. Burmeister T, Gokbuget N, Reinhardt R, Rieder H, Hoelzeri D, Schwartz S. NUP214-ABL1 in adult T-ALL: the GMALL study group experience. Blood 2006.
10. Pui C H, Relling M V, Downing J R. Acute lymphoblastic leukemia. N Engl J Med 2004; 350:1535-48.
11. Druker B J, Guilhot F, O'Brien S G, et al. Five-year follow-up of patients receiving imatinib for chronic myeloid leukemia. N Engl J Med 2006; 355:2408-17.
12. Weisberg E, Manley P W, Breitenstein W, et al. Characterization of AMN107, a selective inhibitor of native and mutant Bcr-Abl. Cancer Cell 2005; 7:129-41.
13. Lee F, Lombardo, L, Camuso, A, et al. BMS-354825 potently inhibits multiple selected oncogenic tyrosine kinases and possesses broad spectrum anti-tumor activities in vitro and in vivo. Proc Am Assoc Cancer Res 2005; 46:159 (abstr 675).
14. Lombardo L J, Lee F Y, Chen P, et al. Discovery of N-(2-chloro-6-methyl-phenyl)-2-(6-(4-(2-hydroxyethyl)-piperazin-1-yl)-2-methylpyrimidin-4-ylamino)thiazole-5-carboxamide (BMS-354825), a dual Src/Abl kinase inhibitor with potent antitumor activity in preclinical assays. J Med Chem 2004; 47:6658-61.
15. Shah N P, Tran C, Lee F Y, Chen P, Norris D, Sawyers C L. Overriding imatinib resistance with a novel ABL kinase inhibitor. Science 2004; 305:399-401.
16. Ottmann O, Martinelli, G, Dombret, H, et al. A phase II study of dasatinib in patients with chronic myeloid leukemia (CML) in lymphoid blast crisis or Philadelphia chromosome-positive acute lymphoblastic leukemia (Ph+ ALL) who are resistant or intolerant to imatinib: The ‘START-L’ CA180015 study. Blood 2005; 106: (abstr 42).
17. Kantarjian H, Giles F, Wunderle L, et al. Nilotinib in imatinib-resistant CML and Philadelphia chromosome-positive ALL. N Engl J Med 2006; 354:2542-51.
18. Graux C, Lafage, M, Dastugue, N, et al. Characterization of 14 NUP214-ABL1 Fusions in T-Cell Acute Lymphoblastic Leukaemia (T-ALL) Exhibits a Genomic Heterogeneity. Blood 2006; 108: (abstr 2075).
19. Nagar B, et al., Crystal Structures of the Kinase Domain of c-Abl in Complex with the Small Molecule Inhibitors PD1173955 and Imatinib (STI-571). Cancer Res. 62, 4236 (2002)).
20. Rowley J D, Potter D. Chromosomal banding patterns in acute nonlymphocytic leukemia. Blood 1976; 47:705-21.
21. Lillington D M, MacCallum P K, Lister T A, Gibbons B. Translocation t(6; 9)(p23; q34) in acute myeloid leukemia without myelodysplasia or basophilia: two cases and a review of the literature. Leukemia 1993; 7:527-31.
22. Alsabeh R, Brynes R K, Slovak M L, Arber D A. Acute myeloid leukemia with t(6; 9) (p23; q34): association with myelodysplasia, basophilia, and initial CD34 negative immunophenotype. Am J Clin Pathol 1997; 107:430-7.
23. Soekarman D, von Lindern M, Daenen S, et al. The translocation (6; 9) (p23; q34) shows consistent rearrangement of two genes and defines a myeloproliferative disorder with specific clinical features. Blood 1992; 79:2990-7.
24. von Lindern M, Breems D, van Baal S, Adriaansen H, Grosveld G. Characterization of the translocation breakpoint sequences of two DEK-CAN fusion genes present in t(6;9) acute myeloid leukemia and a SET-CAN fusion gene found in a case of acute undifferentiated leukemia. Genes Chromosomes Cancer 1992; 5:227-34.
25. Kraemer D, Wozniak R W, Blobel G, Radu A. The human CAN protein, a putative oncogene product associated with myeloid leukemogenesis, is a nuclear pore complex protein that faces the cytoplasm. Proc Natl Acad Sci USA 1994; 91:1519-23.
26. Boer J, Bonten-Surtel J, Grosveld G. Overexpression of the nucleoporin CAN/NUP214 induces growth arrest, nucleocytoplasmic transport defects, and apoptosis. Mol Cell Biol 1998; 18:1236-47.
27. van Deursen J, Boer J, Kasper L, Grosveld G. G2 arrest and impaired nucleocytoplasmic transport in mouse embryos lacking the proto-oncogene CAN/Nup214. Embo J 1996; 15:5574-83.
28. De Keersmaecker K, Bernard, R, Folens, C, et al. Oncogenic Properties of the T-ALL Associated EML1-ABL1 and NUP214-ABL1 Fusion Proteins. Blood 2006; 108: (abstr 1830).
29. Hahn P J. Molecular biology of double-minute chromosomes. Bioessays 1993; 15:477-84.
30. Maurer B J, Lai E, Hamkalo B A, Hood L, Attardi G. Novel submicroscopic extra chromosomal elements containing amplified genes in human cells. Nature 1987; 327:434-7.
31. Ballerini P, Busson M, Fasola S, et al. NUP214-ABL1 amplification in t(5;14)/HOX11L2-positive ALL present with several forms and may have a prognostic significance. Leukemia 2005; 19:468-70.
32. Carlesso N, Frank D A, Griffin J D. Tyrosyl phosphorylation and DNA binding activity of signal transducers and activators of transcription (STAT) proteins in hematopoietic cell lines transformed by Bcr/Abl. J Exp Med 1996; 183:811-20.
33. Ilaria R L, Jr., Van Etten R A. P210 and P190(BCR/ABL) induce the tyrosine phosphorylation and DNA binding activity of multiple specific STAT family members. J Biol Chem 1996; 271:31704-10.
34. Zimmerman et al., Bioorg. Med. Chem. Lett. 7, 187 (1997).
35. Golas et al., Cancer Research (2003) 63, 375-381.
36. Green et al., Preclinical Activity of AZD0530, a novel, oral, potent, and selective inhibitor of the Src family kinases. Poster 3161 presented at the EORTC-NCI-AACR, Geneva Switzerland 28 Sep. 2004.
37. Rosee et al., Cancer Research (2002) 62, 7149-7153.
38. Warmuth et al., Blood, (2003) 101(2), 664-672.
39. O'Hare et al., Clin. Cancer Res (2005) 11(19), 6987-6993.
40. Kimura et al., Blood, 106(12):3948-3954 (2005).

We claim:

1. A method for predicting responsiveness of an individual with a T cell malignancy to treatment with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, which method comprises:
screening a biological sample from said individual for the presence of NUP214-ABL1,
wherein the presence of NUP214-ABL1 in the biological sample indicates that said individual with the T cell malignancy is predicted to be responsive to treatment with N-(2-chloro-6-methylphenyl)-2-[[6-[4-(2-hydroxyethyl)-1-piperazinyl]-2-methyl-4-pyrimidinyl]amino]-5-thiazolecarboxamide.

2. The method of claim 1, wherein the T cell malignancy is T-cell acute lymphocytic leukemia.

3. The method of claim 1, wherein screening for NUP214-ABL1 comprises performing reverse-transcriptase polymerase chain reaction (RT-PCR).

4. The method of claim 1, wherein screening for NUP214-ABL1 comprises performing fluorescence in situ hybridization (FISH).

5. A method of treating an individual suffering from a T cell malignancy, which method comprises:
determining whether the individual harbors NUP214-ABL1; and
administering a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, to the individual who harbors NUP214-ABL1.

6. The method of claim 5, wherein the thiazolecarboxamide or pharmaceutically acceptable salt, hydrate, or solvate thereof is administered at a dosage of greater than 100 mg once daily.

7. The method of claim 5, wherein the thiazolecarboxamide, or a pharmaceutically acceptable salt, solvate, or hydrate thereof, is administered in combination with a second therapy to treat the T cell malignancy in the individual.

8. The method of claim 7, wherein the second therapy is a tubulin stabilizing agent, a farnysyl transferase inhibitor, a BCR-ABL T315I inhibitor, a second protein tyrosine kinase inhibitor, or a combination thereof.

9. The method of claim 7 wherein the second therapy is imatinib, AMN107, PD180970, CGP76030, AP23464, SKI 606, or AZD0530.

10. The method of claim 5, wherein the T cell malignancy is T-cell acute lymphocytic leukemia.

11. The method of claim 5, wherein determining whether the individual harbors NUP214-ABL1 comprises performing reverse-transcriptase polymerase chain reaction (RT-PCR).

12. The method of claim 5, wherein determining whether the individual harbors NUP214-ABL1 comprises performing fluorescence in situ hybridization (FISH).

13. A kit for use in determining a treatment strategy for an individual with a T cell malignancy, comprising:
a means for determining whether the individual harbors NUP214-ABL1; and
instructions for use and interpretation of the kit results.

14. The kit of claim 13, wherein the kit further comprises:
a pharmaceutical composition comprising N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate or solvate thereof in a pharmaceutically acceptable carrier or excipient.

15. The kit of claim 13 further comprising a means for obtaining a biological sample from said individual.

16. The use of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate or solvate thereof for preparing a medicament for the treatment of a patient with a NUP214-ABL1 positive T cell malignancy.
